(19) United States
(12) Patent Application Publication
(10) Pub. No.: US 2022/0031942 A1
(43) Pub. Date: Feb. 3, 2022

(54) NUTRITION PUMP, INFUSION DEVICE, CONTROL VALVE AND LIQUID CONTROL METHOD

(71) Applicant: ZHEJIANG MDKINGDOM TECHNOLOGY CO., LTD., Lishui City, Zhejiang Province (CN)

(72) Inventors: Yajie SONG, Lishui (CN); Weihai JIANG, Lishui (CN); Jianguang TU, Lishui (CN)

(73) Assignee: ZHEJIANG MDKINGDOM TECHNOLOGY CO., LTD., Lishui City, Zhejiang Province (CN)

(21) Appl. No.: 17/427,524

(22) PCT Filed: Mar. 1, 2019
(86) PCT No.: PCT/CN2019/076660

§ 371 Date: Jul. 30, 2021

(30) Foreign Application Priority Data
Jan. 31, 2019 (CN) 201910100869.6

(51) Int. Cl. A61M 5/168; A61M 5/142

(52) U.S. Cl. CPC A61M 5/16827; A61M 2005/1403; A61M 5/142; A61M 5/16813

(57) ABSTRACT

The present disclosure relates to a nutrition pump, an infusion set, a control valve and a method for controlling liquid comprising a pump body and an infusion set, wherein the infusion set comprises a control valve, and the pump body is mounted with a pump wheel; the control valve comprises a valve body and a valve core capable of matingly connected to the valve body, the valve body comprising a first input pipe and a second input pipe for connecting with the liquid to be infused, a cylindrical structure and an output pipe for outputting the liquid to be infused, one end of a coupling shaft is connected to a motor, and the other end is connected to the valve core so that the motor drives the valve core to rotate through the coupling shaft. The disclosure can realize the free switching of the two liquids, eliminate the risk of liquid leakage in the feeding process of the nutrition pump with a low cost and high precision.

### FIELD

The present disclosure relates to the field of medical equipment, in particular to a nutrition pump, an infusion set, a control valve and a method for controlling liquid.

### BACKGROUND

The nutrition pump is a mechanical device used for enteral nutrition infusion to patients, using a disposable enteral nutrition device as a consumable. As an automatic infusion device that accurately controls the infusion speed and volume, the nutrition pump is used for the injection of nutrient solutions to the patient in the clinic. At present, the disposable enteral nutrient solution infusion set is generally used in a single pipe, however, nutrient solutions are usually thick, so that the pipe is prone to blockage during use. Then a double-pipe nutrient solution infusion set appears, by using one pipe for infusion of the nutrient solution, and another for infusion of the cleaning solution, which can effectively prevent pipe blockage and can replenish patients with water. The dual-pipe nutrient solution infusion set currently on the market is used in conjunction with a nutrient pump, and there are two ways to switch between the nutrient solution and the cleaning solution:

Way 1: squeezing one of the pipes for infusing nutrient solution or cleaning solution. This way has the following disadvantages:

1). During the storage process of the nutrient pipe, the Robert clamp is in an open state. During the process of filling the nutrient solution, the Robert clamp must be closed first, otherwise the nutrient solution and the cleaning solution will leak. Some of them partially added a check valve to prevent the risk of liquid leakage caused by human mistakes, and it increased the cost of pipe production (the accuracy of the check valve is very high).

2). Some nutrition pumps do not have a sensor for detecting the position of the liquid stop clamp, and rely only on the mechanical structure to ensure the liquid stop of the squeezed pipe, without considering the wear and mechanical failure after long-term use.

Way 2: using a three-way valve, connecting a shaft to a valve core, and detecting the positions of the shaft and the valve core for infusing nutrient solution or cleaning solution. The disadvantages of this solution are the cost for manufacturing the valve is high, the rotation angle and stop position of the shaft need to be controlled very accurately, which increases the cost of the enteral nutrition device and the nutrition pump.

### SUMMARY

The object of the present disclosure is to completely solve the problem of liquid leakage during the infusion of nutrient solution and cleaning solution in the nutrient pump with convenient mounting and simple operation.

The object of the present disclosure is achieved through the following technical solutions:

An inventive concept of the present disclosure is to provide a nutrition pump comprising a pump body and an infusion set, in which the infusion set comprising a control valve, and the pump body is mounted with a pump wheel, two DC motors and a coupling shaft; wherein the control valve comprises a valve body and a valve core capable of matingly connecting with the valve body, the valve body comprises a first input pipe for connecting with the liquid to be transported and a second input pipe, a cylindrical structure, and an output pipe for outputting the liquid to be transported, the cylindrical structure is composed of a first cylindrical structure and a second cylindrical structure arranged side by side in parallel; the valve core comprises a first valve core capable of being exactly accommodated in the first cylindrical structure and rotating in the first cylindrical structure, and a second valve core capable of being exactly accommodated in the second cylindrical structure and rotating in the second cylindrical structure; one end of the coupling shaft is connected to the motor, and the other end is connected to the valve core so that the motor drives the valve core to rotate through the coupling shaft; the side wall of the first cylindrical structure is provided with a first through hole communicating with the first input pipe and a second through hole communicating with the output pipe, respectively, and correspondingly, the side wall of the second cylindrical structure is provided with a third through hole communicating with the second input pipe and a fourth through hole communicating with the output pipe, respectively; the side wall of the first valve core is provided with a first hole and a second hole that communicate with each other, and the side wall of the second valve core is provided with a third hole and a fourth hole that communicate with each other; when the two through holes on the cylindrical structure and the two holes on the valve core are aligned one by one, the liquid enters the output pipe through the valve core, and the valve core is opened; when the two through holes on the cylindrical structure are staggered with the two holes on the valve core, the liquid in the input pipe cannot enter the output pipe, and the valve core is closed.

Further, the first valve core is provided with a bent first communicating pipe that communicates the first hole and the second hole to allow liquid to pass through; and the second valve core is also provided with a bent second communicating pipe connecting the third hole and the fourth hole to allow the liquid to pass through.

Further, the first hole and the second hole on the first valve core are two independent through holes distributed at 90 degrees, and the third hole and the fourth hole on the second valve core are also two independent through holes distributed at 90 degrees; correspondingly, the first through hole and the second through hole on the first cylindrical structure are two independent through holes distributed at 90 degrees, and the third through hole and the fourth through hole on the second cylindrical structure are also two independent through holes distributed at 90 degrees.

Further, the centers of one end surface of the first valve core and of the second valve core are each provided with a flat groove; the first cylindrical structure and the second cylindrical structure are both horizontally arranged in a front-to-rear direction, and the first input pipe and the second input pipe are respectively arranged vertically on the top of the first cylindrical structure and the top of the second cylindrical structure, and respectively communicate with the first through hole and the third through hole; the output pipe is vertically arranged at the lower centers of the first cylindrical structure and the second cylindrical structure, and the top end of the output pipe is located between the first cylindrical structure and the second cylindrical structure.

Further, the first cylindrical structure and the second cylindrical structure are both cylinder-shaped structures with one end opened and the other closed, and at least one annular groove is provided on the inner walls of the first cylindrical structure and the second cylindrical structure; the outer peripheries of the first valve core and the second valve core are both provided with an annular protruding strip matched with the annular groove so that the valve core can be fixedly connected in the cylindrical structure and can rotate freely in the cylindrical structure; the infusion set further comprises a first upper pipe, a second upper pipe, and a lower pipe, and the first input pipe in the control valve is connected to the first upper pipe used for transporting liquid, and the second upper pipe is connected to the second input pipe used for transporting liquid, and the output pipe is connected to the down pipe to output the liquid from the down pipe.

Further, the first cylindrical structure and the second cylindrical structure are each provided with a first notch at the openings on the walls, and the first valve core and the second valve core are each provided with a second notch at the openings on the walls, and the first notchs on the first cylindrical structure and the second cylindrical structure are respectively aligned with the second notchs on the first valve core and the second valve core in the initial state.

Further, the second notch and the flat groove are both located on the front side of the valve core, and the second notch communicates with the flat groove and has a depth smaller than that of the flat groove; the valve body further comprises a horizontally arranged flat plate, one end of the flat plate is fixedly connected to the outer peripheries of the first input pipe and the second input pipe and extends backward to the outer sides of the first cylindrical structure and the second cylindrical structure, and the bottom surface of the flat plate is fixedly connected to the top surfaces of the first cylindrical structure and the second cylindrical structure; the lower end of the flat plate located above the output pipe extends downward and is fixedly connected to the top of the output pipe, and the upper end of the output pipe is located between the first cylindrical structure and the second cylindrical structure and is fixedly connected to the two cylindrical structures so that the control valve is integrated as a whole.

Further, the coupling shaft comprises a main body, the top end surface of the main body is detachably connected to the motor, a positioning block protruding from the bottom end surface is transversely provided in the center of the bottom end surface of the main body, and one end of the positioning block extends outward to outer side the body, and a shaft is fixed on the lower end surface of the positioning block.

Further, the body comprises a front surface and a back surface, the upper end of the front surface is provided with a mounting groove for mounting the control valve, and the back surface is provided in a place corresponding to the mounting groove with two circular bosses for placing the coupling shaft, the centers of each boss are provided with a fixing hole for communicating with the mounting groove, and the shaft of the coupling shaft passes through the fixing hole from the back of the body and connects with the valve core in the mounting groove.

Further, each of the bosses is provided with two center-symmetric or axisymmetric right-angle convex surfaces, the edges at the right angles of the right-angle convex surfaces are flush with the inner side of the fixing hole.

Further, the shaft is arranged in the positioning hole, the positioning block is placed on the upper end surface of the boss and can be rotated on the upper end surface of the boss with the shaft as a rotation axis, and the two right-angle surfaces of the right-angle convex surface enable the positioning block only to be rotated clockwise or counterclockwise within 90 degrees.

Further, a sensor is provided on the outer side of the two bosses, which can detect the position of the coupling shaft. Since the coupling shaft is connected to the valve core, the state of the valve core (opened/closed) can be detected.

Further, when the positioning block enters the sensor, exactly one side of the positioning block contacts a right-angle side surface of the right-angle convex surface.

Further, the walls on both sides of the lower end of the mounting groove are provided with arc-shaped fixing grooves that can accommodate the first cylindrical structure and the second cylindrical structure exactly, so that the outer side of the first cylindrical structure and the outer side of the second cylindrical structure are exactly clamped in the fixing grooves on both sides when the control valve is in the mounting groove.

Another inventive concept of the present disclosure is to provide an infusion set as described above.

Another inventive concept of the present disclosure is to provide a control valve as described above.

The last inventive concept of the present disclosure is to provide a method of liquid control, the method is as follows: mounting a valve core inside a cylindrical structure with a second notch on the valve core aligned with a first notch of the cylindrical structure; placing a positioning block of a coupling shaft on a boss, and making a shaft of the coupling shaft pass through a fixing hole from the back of a pump body into a mounting groove and connect with the valve core in the mounting groove, connecting two DC motors to the corresponding coupling shafts respectively so that the DC motors drive the corresponding valve cores through the corresponding coupling shafts to rotate synchronously inside the cylindrical structure, so as to realize the alternate output of the nutrient solution and the cleaning solution; specifically when the nutrient solution is needed, opening the valve core corresponding to the nutrient solution and making the nutrient solution pass through the valve core and enter an output pipe; when the cleaning solution is needed, first closing the valve core corresponding to the nutrient solution, and then opening the cleaning solution valve core so that the cleaning solution enters the output pipe through the valve core. The positioning block will enter and exit the detection sensor during its rotation so that the signal is detected by the sensor and transmitted to the circuit board for control; if a mistake occurs, an alarm will occur.

The Beneficial Effects of the Present Disclosure are as Follows:

When the valve core and the valve body of the nutrition pump of the present disclosure are mounted, the gaps of the two valve cores correspond to the gaps of the valve body, and at this time, one of the valve cores is in a closed state and the other is in an opened state. There will be no mistakes in mechanical or manual assembly, which fundamentally eliminates the problem of liquid leakage. And in the process of use: the program is controlled to close the valve core first, and then open the valve core that needs to be opened, so the problem of liquid leakage is also eliminated during use. In addition, the two liquids can be switched freely, with low cost, high precision and no risk of liquid leakage.

With two cylindrical structures and two valve cores in the control valve structure, the inlet and outlet of the two liquids are not the same, and basically no mixing, contamination, etc. will occur.

Mistakes can be prevented to a large extent by arranging ingeniously structured bosses, right-angle convex surfaces, positioning blocks, shafts, and the coordination of these components. In addition, the cooperation among circuit boards containing control chips or single-chip microcomputers, sensors and positioning blocks extending outwards makes the rotation of the two valve cores without any mistakes, or even errors, and the accuracy is very high. In addition, accurate alignment at the beginning is ensured by aligning the second notch on the valve core with the first notch on the cylindrical structure in the initial state, avoiding the risk of errors in the later stage. Then the possible occurrence of liquid leakage is avoided.

The control valve in the present disclosure is highly integrated and forms a solid organic whole. The arrangement of the flat plate not only increases the integration of the control valve, but also can be used as a hand-held plate for users to disassemble and assemble the control valve, and the use effect is greatly increased.

In addition, the infusion set in the present disclosure is used as a consumable part, each part of the infusion set is convenient to assemble, and the infusion set and the pump body are easy to disassemble and assemble, and the use effect is good.

The accuracy of the rotation angle and the stopping position required in the disclosure will not need to be very precise, so the cost can be further reduced, and the occurrence of errors or mistakes will be avoided, thereby avoiding the possible occurrence of liquid leakage.

### BRIEF DESCRIPTION OF DRAWINGS

FIG. 1 is a schematic diagram of the sectional structure of the control valve according to an example of the present disclosure;

FIG. 2 is a schematic diagram of the three-dimensional structure of the control valve according to an example of the present disclosure;

FIG. 3 is a schematic diagram of the three-dimensional structure of the valve body;

FIG. 4 is a top view of the valve body;

FIG. 5 is a right side view of the valve body;

FIG. 6 is a B-B line sectional view in FIG. 5;

FIG. 7 is an A-A line sectional view in FIG. 5;

FIG. 8 is a schematic diagram of the three-dimensional structure of the first valve core;

FIG. 9 is a top view of the first valve core;

FIG. 10 is an A-A line sectional view in FIG. 9;

FIG. 11 is a B-B line sectional view in FIG. 9;

FIG. 12 is a schematic diagram of the structure of the infusion set;

FIG. 13 is a schematic diagram of another structure of the infusion set;

FIG. 14 is a schematic diagram of the main view structure of the nutrition pump;

FIG. 15 is a schematic diagram of the front view of the pump body;

FIG. 16 is a schematic diagram of the three-dimensional structure of the front of the pump body;

FIG. 17 is a schematic diagram of the three-dimensional structure of the back of the pump body;

FIG. 18 is a schematic diagram of the structure of the coupling shaft;

FIG. 19 is a longitudinal sectional rear view of the pump body;

FIG. 20 is a schematic diagram of the connection between the coupling shaft and the sensor;

FIG. 21 is a schematic diagram of the cross-sectional structure of the infusion set.

In the figure, 1, pump body; 2, mounting groove; 3, pipe groove; 4, fixing hole; 5, boss; 6, right-angle convex surface; 7, U-shaped groove; 8, circuit board; 9, fixing groove; 10, control valve; 11, first upper pipe; 12, second upper pipe; 13, nutrient solution; 14, cleaning solution; 15, rubber pipe; 16, first two-way interface; 17, first pipe; 18, Y-shaped three-way interface; 19, second pipe; 20, joint; 21, liquid stop clamp; 22, third pipe; 23, second two-way interface; 24, piercer; 25, down pipe; 26, bearing; 27, coupling shaft; 28, body; 29, positioning block; 30, shaft; 31, flat head; 32, bubble detection device; 33, pressure detection device; 34, pump wheel; 35, motor; 100, first valve core; 101, second valve core; 102, first communicating pipe; 103, second communicating pipe; 104, first hole; 105, second hole; 106, annular protruding strip; 107, second notch; 108, flat groove; 111, first cylindrical structure; 112, second cylindrical structure; 113, first input pipe; 114, second input pipe; 115, output pipe; 116, flat plate; 117, convex edge; 118, annular groove; 119, first notch; 120, first through hole; 121, third through hole; 122, second through hole; 123, fourth through hole; 124, baffle.

### DETAILED DESCRIPTION

The technical solutions in the examples of the present disclosure are described in the following clearly and completely. Obviously, the described examples are part of the examples of the present disclosure, rather than all of them. The detailed description of the examples of the present disclosure provided below is not intended to limit the scope of the claimed present disclosure, but merely represents selected examples of the present disclosure.

In the present disclosure, any description referring to the “cylindrical structure” refers to the first cylindrical structure and/or the second cylindrical structure; any description referring to the “input pipe” refers to the first input pipe and/or the second input pipe; any description referring to “valve core” refers to the first valve core and/or the second valve core; any description referring to “through hole” refers to the first through hole, the second through hole, the third through hole and/or the fourth through hole. If there are specific restrictions in the front, it may refer to the first through hole and/or the second through hole, or the third through hole and/or the fourth through hole, which is specifically determined by the previous limitation; any description referring to “hole” refers to the first hole, the second hole, the third hole, and/or the fourth hole. If there are specific restrictions in the front, it may refer to the first hole and/or the second hole, or the third hole and/or the fourth hole, which is specifically determined by the previous limitation.

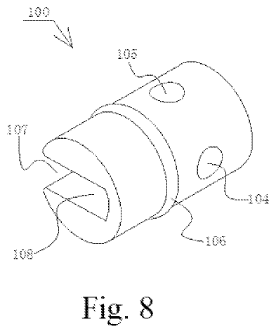

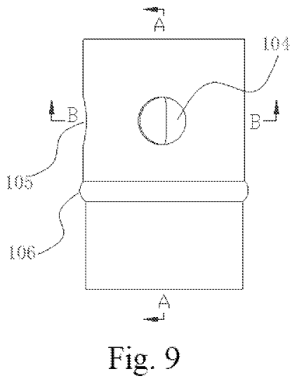

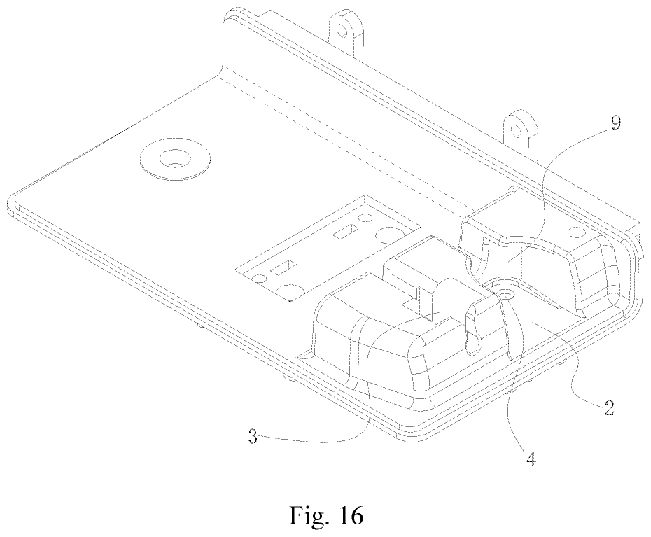

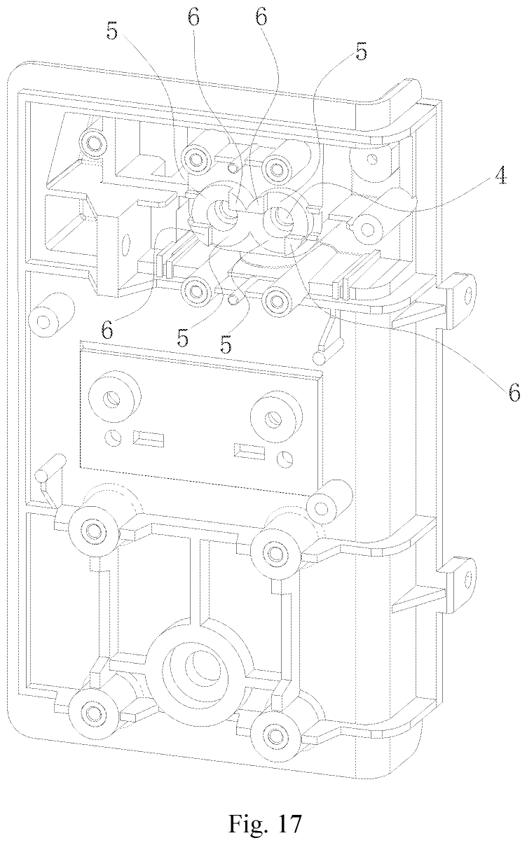

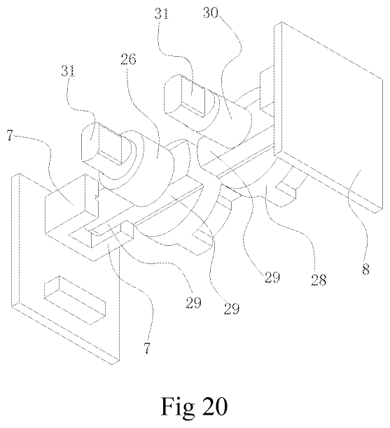

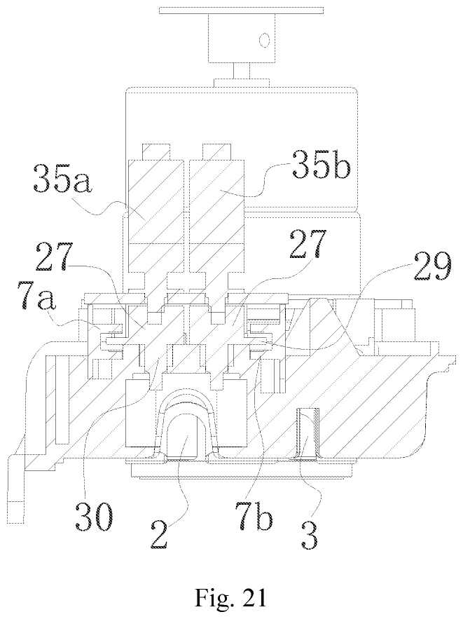

The following is a further explanation with reference to the accompanying drawings. Since the structure of the second valve core is exactly the same as the first valve core, only the schematic diagram of the structure of the first valve core is given in FIGS. 8 and 9 with that of the second valve core omitted. FIG. 16 is a schematic diagram of the three-dimensional structure of the pump body (seen from the front of the pump body); FIG. 17 is a schematic diagram of the three-dimensional structure of the pump body (seen from the back of the pump body), without the coupling shaft 27 and the motor and the like. FIG. 20 is a schematic diagram of the connection between the coupling shaft 27 and the sensor, in which a bearing 26 is sleeved on the shaft 30 on the left, and the bearing 26 is not shown on the shaft 30 on the right; the control valve is not shown in FIG. 21.

### EXAMPLES

### Example 1

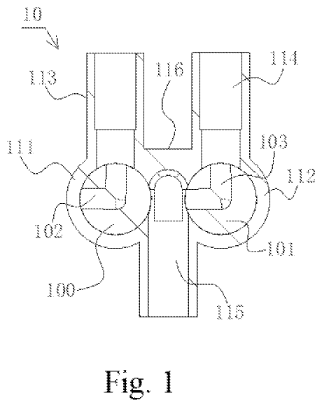

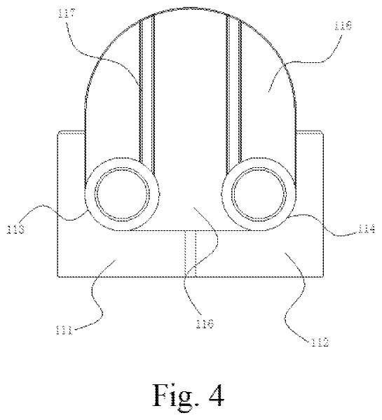

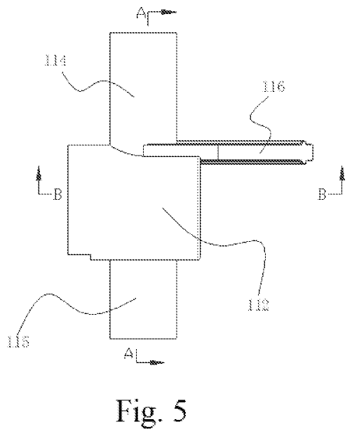

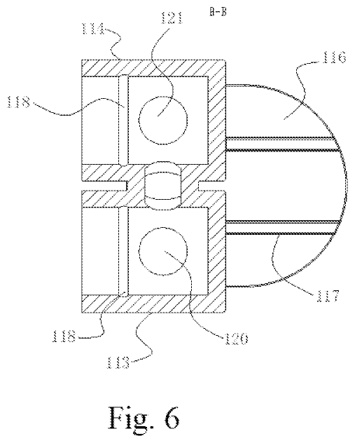

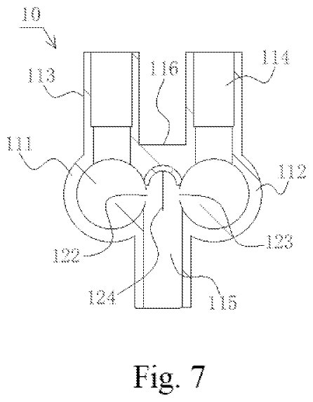

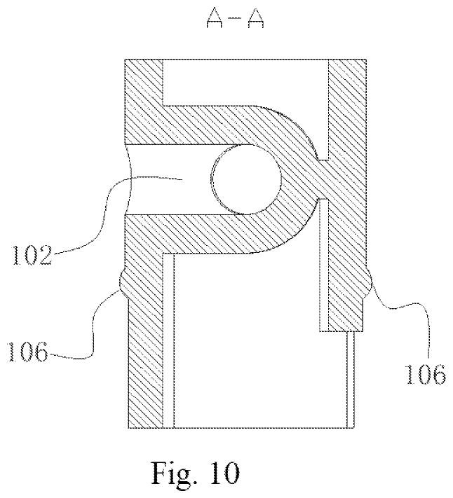

A control valve applied to a nutrition pump is shown in FIGS. 1-11. The control valve 10 comprises a valve body and a valve core that can be mated connected to the valve body. The valve body comprises a first input pipe 113 and a second input pipe 114, output pipe 115, and a first cylindrical structure 111 and a second cylindrical structure 112 arranged side by side in parallel; the valve core comprises a first valve core 100 that can be exactly accommodated in the first cylindrical structure 111 and a second valve core 101 that can be exactly accommodated in the second cylindrical structure 112; the inner walls of the cross section of the first cylindrical structure 111 and the second cylindrical structure 112 are both circular-shaped, as are the outer walls of the section of the first valve core 100 and the second valve core 101, which enables the valve core to rotate freely in the cylindrical structure.

The side wall of the first cylindrical structure 111 is provided with a first through hole 120 communicating with the first input pipe 113 and a second through hole 122 communicating with the output pipe 115. Accordingly, the side wall of the second cylindrical structure 112 is provided with a third through hole 121 communicating with the second input pipe 114 and a fourth through hole 123 communicating with the output pipe 115 respectively.

The first valve core 100 is exactly accommodated and can rotate in the first cylindrical structure 111; and the second valve core 101 is exactly accommodated and can rotate in the second cylindrical structure 112.

The side wall of the first valve core 100 is provided with a first hole 104 and a second hole 105 that communicate with each other, and the side wall of the second valve core 101 is provided with a third hole and a fourth hole that communicate with each other.

When the two through holes on the first cylindrical structure 111 and the two holes on the first valve core 100 are aligned one by one, the liquid in the first input pipe 113 enters the output pipe 115 through the first valve core 100 to allow the valve core to be opened; when the two through holes on the first cylindrical structure 111 are not aligned with the two holes on the first valve core 100, the liquid in the first input pipe 113 cannot enter the output pipe 115 to allow the valve core to be closed. Correspondingly, when the two through holes on the second cylindrical structure 112 and the two holes on the second valve core 101 are aligned one by one, the liquid in the second input pipe 114 enters the output pipe 115 through the second valve core 101 to allow the valve core to be opened; when the two through holes on the second cylindrical structure 112 and the two holes on the second valve core 101 are not aligned one by one, the liquid in the second input pipe 114 cannot enter the output pipe 115 to allow the valve core to be closed.

The first valve core 100 is provided with a bent first communicating pipe 102 that communicates the first hole 104 and the second hole 105 to allow the liquid to pass through; and the second valve core 101 is also provided with a bent second communicating pipe 103 that communicates the third hole and the fourth hole to allow the liquid to pass through.

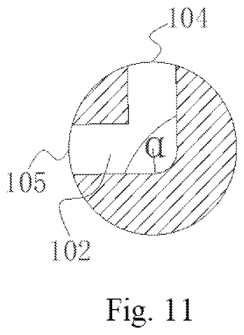

The first hole 104 and the second hole 105 on the first valve core 100 are two independent through holes distributed at 90 degrees, and the third hole and the fourth hole on the second valve core 101 are also two independent through holes distributed at 90 degrees. As shown in FIG. 11, the two holes correspond to the two ends distributed in a quarter arc.

Correspondingly, the first through hole 120 and the second through hole 122 on the first cylindrical structure 111 are two independent through holes distributed at 90 degrees, and the third through hole 121 and the fourth through hole 123 on the second cylindrical structure 112 are also two independent through holes distributed at 90 degrees;

In this way, the valve core and the holes on the cylindrical structure can be made one-to-one.

The bending angle α of the communicating pipe is 90 to 130 degrees, such as 90 degrees, 95 degrees, 100 degrees, etc., when the liquid flows into the output pipe 115 from the bend of the communicating pipe, it flows horizontally or obliquely downward and passes through smoothly without producing any effusion. If the angle is too small, effusion will be produced, or the flow rate will be slow; if the angle is too large, the flow rate will be too large and the effect will be difficult to control.

The two holes on the valve core are opened on the wall, and the two holes are distributed at 90 degrees. Compared with the way of up and down distribution, there will be a bending process from one hole to the other hole, that is, the communicating pipe bent inside has an important buffering effect on the flow rate of body fluid, especially before it entering the output pipe, controlling the flow rate makes it easier to control the overall liquid flow and also allows the liquid to flow smoothly along the output pipe as it enters the output pipe. For example, the liquid will not be splashed to the second through hole or other places, the consistency of effect of falling speed of all body fluids is good, and it is not easy to have any mixing with another liquid flowing down later. The liquid moves completely downwards in the output pipe, and the inner wall of the output pipe is now basically liquid-phobic, so no liquid will be retained on the wall, so the mixing of the two liquids is basically eliminated from all aspects. That is, the occurrence of liquid leakage is effectively prevented.

The first cylindrical structure 111 and the second cylindrical structure 112 are both horizontally arranged in the front-to-rear direction, and the first input pipe 113 and the second input pipe 114 are respectively arranged vertically on the top of the first cylindrical structure 111 and the top of the second cylindrical structure 112, the first through hole 120 is provided on the top of the first cylindrical structure 111, and the third through hole 121 is provided on the top of the second cylindrical structure 112, so the first input pipe 113 and the second input pipe 114 respectively communicate with the first through hole 120 and the third through hole 121.

The output pipe 115 is vertically arranged at the lower center of the first cylindrical structure 111 and the second cylindrical structure 112, and the top end of the output pipe 115 is located between the first cylindrical structure 111 and the second cylindrical structure 112;

The first cylindrical structure 111 is provided with the second through hole 122 where it is connected to the output pipe 115, and the second cylindrical structure 112 is provided with the fourth through hole 123 where it is connected to the output pipe 115.

The output pipe 115 is provided with a first hole 104 and a second hole respectively communicating with the second through hole 122 and the fourth through hole 123 where it is connected to the first cylindrical structure 111 and the second cylindrical structure 112, so that the liquid coming out of the first cylindrical structure 111 or the second cylindrical structure 112 can directly enter the output pipe 115.

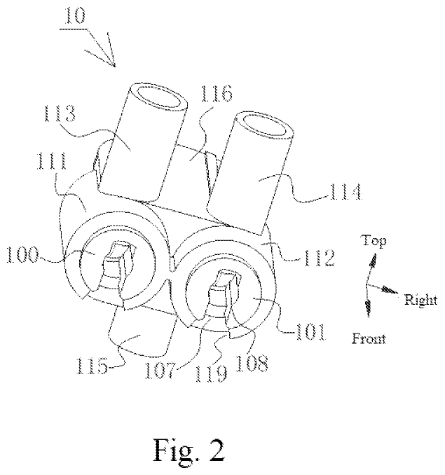

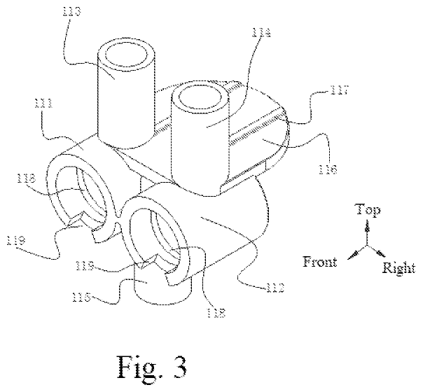

The first cylindrical structure 111 and the second cylindrical structure 112 are both cylinder-shaped structures with one end opened and the other closed, that is, the front end opened and the rear end closed. See FIG. 2-3, at least one ring of annular groove 118 is provided on the inner wall of the first cylindrical structure 111 and the second cylindrical structure 112;

The outer peripheries of the first valve core 100 and the second valve core 101 are both provided with an annular protruding strip 106 adapted to the annular groove 118, so that when the first valve core 100 is located in the first cylindrical structure 111, the protruding strip 106 is exactly located in the annular groove 118, so the first valve core 100 can only be fixed but also rotated in the first cylindrical structure 111. Similarly, the second valve core 101 can only be fixed but also rotated in the second cylindrical structure 112.

The first cylindrical structure 111 and the second cylindrical structure 112 are each provided with a first notch 119 at the openings on the walls, and the first valve core 100 and the second valve core 101 are each provided with a second notch 107 at the openings on the walls. In the initial state, the second notch 107 of the first valve core 100 is aligned with the first notch 119 on the first cylindrical structure 111, and the second notch 107 of the second valve core 101 is aligned with the first notch 119 on the second cylindrical structure 112 similarly.

As a further preferred embodiment, a baffle 124 is vertically arranged at the center of the upper end of the output pipe 115, two surfaces of which face the second through hole 122 and the fourth through hole 123, respectively, to prevent liquid from one cylindrical structure from splashing into the outlet of the other. If the baffle 124 is not added, the liquid from one cylindrical structure may splash to the outlet of the other cylindrical structure when the flow rate is high, using more time than the normal time of the liquid falling. It may even cause the liquid to be stuck at another outlet, which is very likely to be mixed with the liquid that subsequently comes out of another cylindrical structure. Therefore, a baffle 124 can be provided to prevent the above-mentioned situation from occurring, and the possibility of mixing of two liquids can be further avoided, and the use effect is better. The baffle 124 is simple to set, simple in process, and low in cost.

As a further preferred example, a flat groove 108 is provided in the centers of one end surface of the first valve core 100 and the second valve core 101. The second notch 107 and the flat groove 108 are located in the same direction, that is, both are located on the front side of the valve core. The front and rear directions are shown in FIG. 2, and the second notch 107 communicates with the flat groove 108. The second notch 107 has a depth smaller than that of the flat groove 108, so that the coupling shaft 27 can be more firmly connected to the valve core, and the overall rotation effect is more accurate.

The valve body also comprises a flat plate 116 arranged horizontally, that is, parallel to the surface where the first cylindrical structure 111 and the second cylindrical structure 112 are located. One end of the flat plate 116 is fixedly connected to the outer peripheries of the first input pipe 113 and the second input pipe 114 and extends backward to the outer sides of the first cylindrical structure 111 and the second cylindrical structure 112. The bottom surface of the flat plate 116 is fixedly connected to the top surface or upper end surface of the first cylindrical structure 111 and the second cylindrical structure 112. That is, where the input pipe is connected to the cylindrical structure, the flat plate 116 is fixed to the two input pipes as well as the two cylindrical structures at the same time, and the flat plate 116 is fixed to the top surface of the cylindrical structure where it passes through the cylindrical structure.

In addition, the lower end of the flat plate 116 located above the output pipe 115 extends downward and is fixedly connected to the top of the output pipe 115. As described above, the upper end of the output pipe 115 is fixedly connected to two cylindrical structures, and the top of the output pipe 115 then fixedly connected to the plate 116, the arrangement of the plate 116 is not only convenient for the user to mount or remove the control valve 10, but more importantly, the plate 116 integrates two cylindrical structures and two input pipes into a whole, making the use effect greatly increased.

Preferably, the flat plate 116 is provided with an edge 117 parallel to the axis of the first cylindrical structure 111 or the second cylindrical structure 112, which can enhance the strength of the flat plate 116 and also provide a point of force for the user to hold and prevents it from slipping out of the user's hand.

In this example, the two valve cores are preferably valve cores with exactly the same structure. In the initial state, as shown in FIG. 1, the two valve cores can be mounted in the same direction, that is, the two valve cores are placed exactly the same, in which the two holes in a valve core are aligned with the two through holes in the corresponding cylindrical structure one by one, as shown on the right side of FIG. 1, the liquid can be connected to and enter the output pipe 115 from the input pipe; then the hole in the other valve core is staggered with the through hole on the corresponding cylindrical structure, as shown on the left side of FIG. 1, that is, liquid cannot enter the output pipe 115 from the input pipe, and only rotating the valve core 90 degrees clockwise can allow the liquid into the output pipe 115 to achieve a connected state.

Preferably in FIG. 1, the first notches 119 on the two cylindrical structures are located at the lowest end of the opening wall, and the second notches 107 on the two valve cores are also located at the lowest end of the opening wall, then the first notch 119 is aligned with the second notch 107, that is, in the original state of the factory, the valve core is rotated to make the second notch 107 at the bottom end to align with the first notch 119 when mounted. At this time, it is a state that one valve core is connected (right side connected) and another valve core is closed (left side closed); if two valve cores rotate 90 degrees clockwise at the same time, on the contrary, that is, the right valve core is closed and the left side is opened. Then when it is rotated 90 degrees counterclockwise at the same time, it will return to the state that the right side is connected the left side is closed. Finally, alternation of the two liquids can be realized.

### Example 2

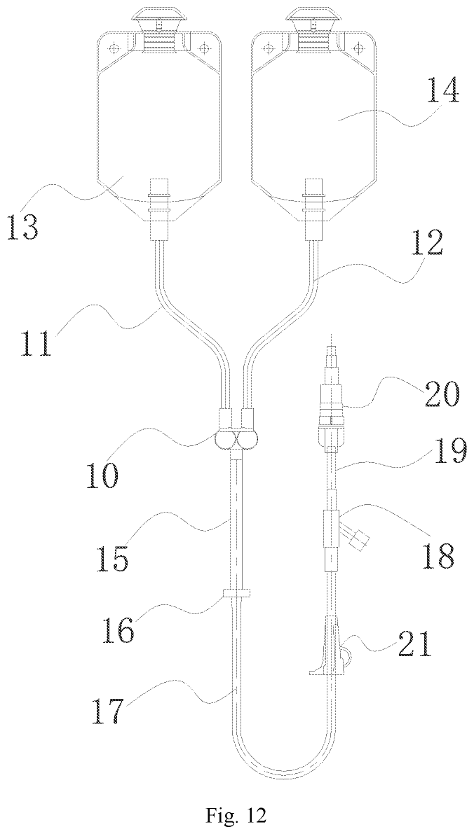

An enteral nutrient solution infusion device, as shown in FIG. 12, which comprises the control valve 10 described in Example 1, in which the first input pipe 113 is connected to a first upper pipe 11 for transporting liquid, a second input pipe 114 is connected to a second upper pipe 12 for transporting liquid, and an output pipe 115 is connected to a down pipe 25 to output the liquid from the down pipe 25, and the down pipe 25 is connected to a connector 20 used for connecting to the human body, and the connector 20 is connected to the human body to allow liquid to enter the human body. The connector 20 is preferably a Luer connector, which is convenient to use and has a good effect.

The first upper pipe 11 and the second upper pipe 12 can be connected to the nutrient solution 13 and the cleaning solution 14 respectively so that the two liquids enter the control valve 10 through their respective pipes.

The down pipe 25 comprises a rubber pipe 15, a first pipe 17 and a second pipe 19, in which one end of the rubber pipe 15 is connected to the output pipe 115, and the other end is connected to the first pipe 17 through a first two-way interface 16, the first pipe 17 is connected to the second pipe 19 by a Y-shaped three-way interface 18, and a liquid stop clamp 21 is also connected to the second pipe 19. The setting of the rubber pipe can make the feeding speed of the nutrition pump more stable.

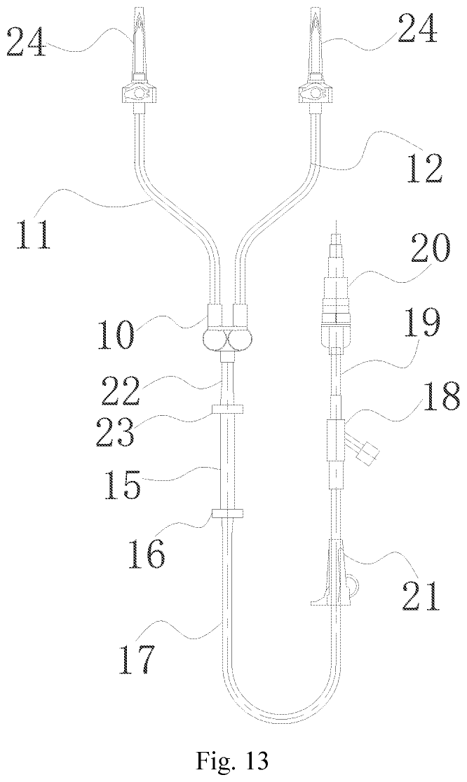

As one of the embodiments, as shown in FIG. 13, the down pipe 25 further comprises a third pipe 22, in which one end of the third pipe 22 is connected to the output pipe 115, and the other end communicates with the rubber pipe 15 through a second two-way interface 23. The method for connection behind the rubber pipe 15 is the same as above, and will not be repeated here.

In this example, the nutrient solution bag 13 and the cleaning solution bag 14 can be replaced with two piercers 24, as shown in FIG. 13, the piercer 24 can be used with different medical solutions, nutrient solutions 13 and/or cleaning solutions 14, etc., to achieve the infusion of different medical solutions.

In this example, the infusion set is fully equipped. When in use, it only needs to be connected to a motor and a circuit board 8. For example, two motors are prepared, the output shafts of which are connected to the flat groove 108 on the first valve core 100 and the second valve core 101, respectively, that is, the part of the output shaft connected to the flat groove 108 is also flat-shaped so that it can be exactly accommodated in the flat groove 108. The circuit board can be integrated with a control chip or connected to a processor/controller to achieve control. When rotating, the rotation of the motor drives the output shaft of the motor to rotate, which in turn drives the rotation of the valve core. The rotation of the valve core can realize the alignment or stagger of the holes on the valve core and the through holes on the cylindrical structure, and realize the connection (control valve opened) or blockage (control valve closed) of the liquid.

### Example 3

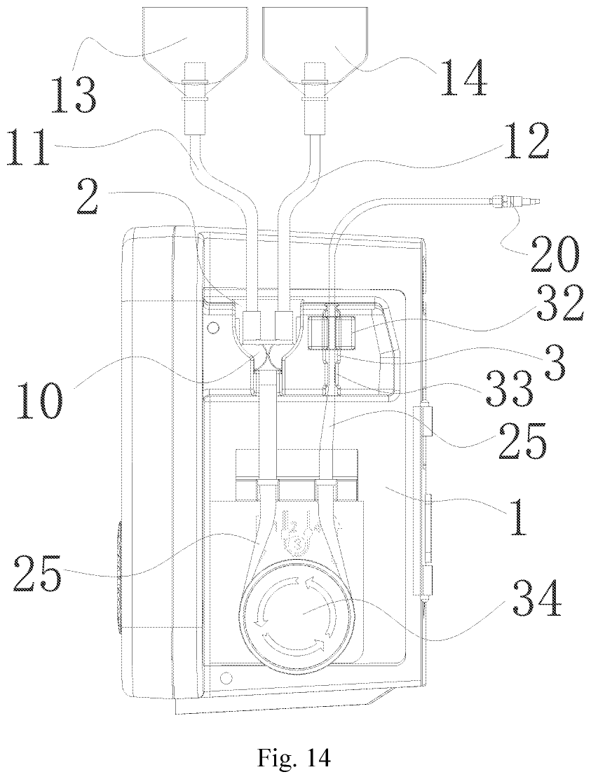

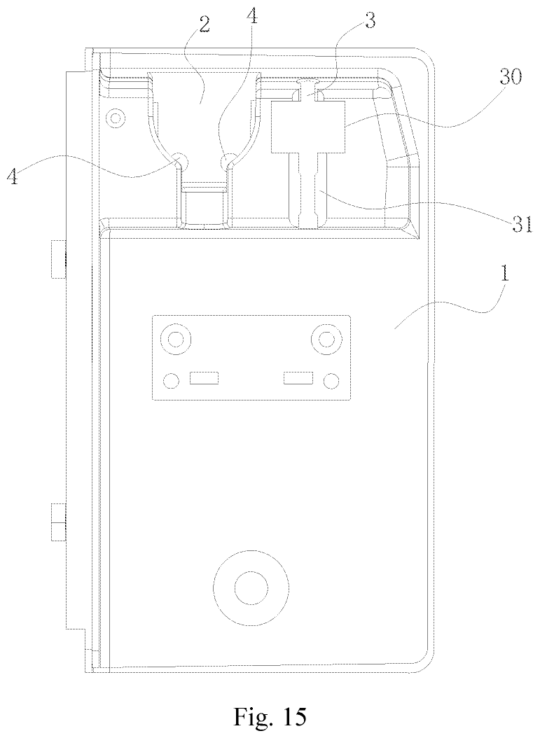

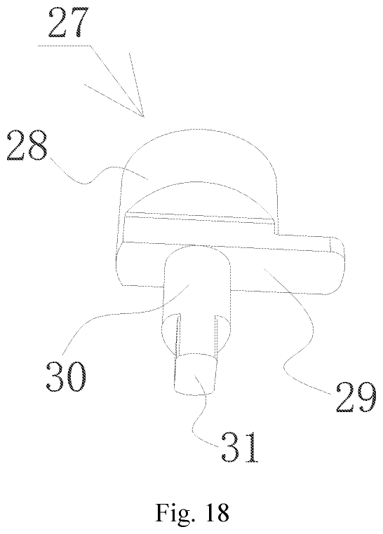

As shown in FIGS. 14-20, a nutrition pump comprises a pump body 1 and the infusion set as described in Example 2.

The infusion set comprises the control valve 10 as described in Example 1.

A pump wheel 34 is mounted on the pump body 1, and the down pipe bypasses the pump wheel and passes out of the pump body 1.

Two motors and two coupling shafts 27, in which one end of the coupling shaft 27 is connected to the first valve core 100 to make the coupling shaft 27 drive the valve core to rotate, and the other end is connected to one of the motors to make the motor drive the coupling shaft 27 to rotate, and similarly, another coupling shaft 27 is connected to the second valve core 101 and another motor to realize that the motor drives the valve core to rotate.

The coupling shaft 27 comprises a body 28, the top surface of the body 28 is fixedly connected to the motor, the center of the bottom end surface of the body 28 is transversely provided with a positioning block 29 protruding from the bottom end surface, and one end of the positioning block 29 extends outwards to the outer side of the body 28, the bottom end surface and two side surfaces of the positioning block 29 are both horizontal or vertical surfaces. The bottom end surface of the positioning block 29 is fixed with a shaft 30. The free end of the shaft 30 is a flat head 31 used for extending into the flat groove 108 of the valve core to drive the valve core to rotate. The mating manner of the flat head 31 and the flat groove 108 can easily drive the rotation.

The pump body 1 comprises a front surface and a back surface. The upper end of the front surface is provided with a mounting groove 2 for mounting the control valve 10 and a pipe groove 3 for placing a down pipe 25 and the like. The lower end of the front surface is provided with a pump wheel 34. The pipe groove 3 is used to place the down pipe 25 and is provided with a bubble detection device 32 and a pressure detection device 33 for detecting bubbles and pressure generated by the liquid flowing through the down pipe. The bubble detection device 32 can be a bubble sensor, and the pressure detecting device 33 can be a pressure sensor.

The back side is provided with two circular bosses 5 for placing the coupling shaft 27 at the place corresponding to the mounting groove 2, and the center of each boss 5 is provided with a fixing hole 4 communicating with the mounting groove 2. The shaft 30 of the coupling shaft 27 can penetrate into the mounting groove 2 through the fixing hole 4 from the back side, and be connected to the control valve 10 in the mounting groove 2.

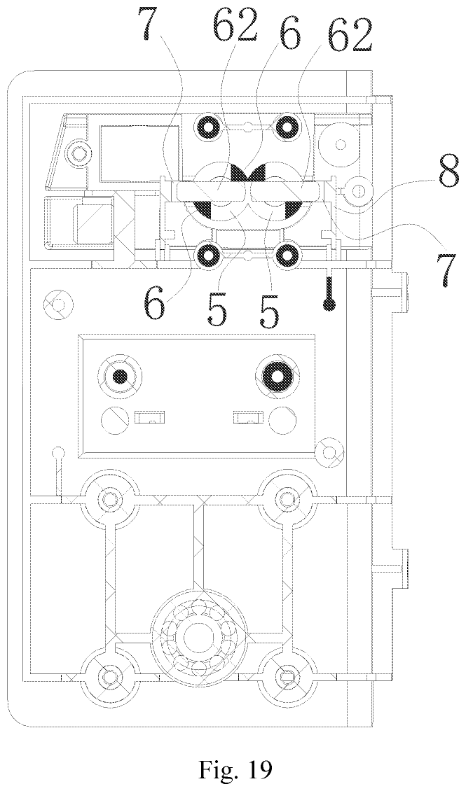

Each of the bosses 5 is provided with two center-symmetric or axisymmetric right-angle convex surfaces 6, as shown in FIGS. 17 and 19, the edges of the right-angle vertices of the right-angle convex surfaces 6 (that is, the right-angle edges connected to the bosses 5 at the right angle) are flush with the inner side surface of the fixing hole 4.

When the shaft 30 extends into the positioning hole, the positioning block 29 is placed on and can rotate on the upper end surface of the boss 5 with the shaft 30 as the rotation axis, and the two right-angle edges of the right-angle convex surface 6 has a limiting effect, so that the positioning block 29 can only rotate clockwise or counterclockwise within a range of 90 degrees, that is, the maximum rotation angle of which is 90 degrees. The rotation range of the valve core is limited, and the accuracy of rotation is increased.

Preferably, a bearing 26 is provided in the positioning hole. The height of the bearing 26 is consistent with the depth of the positioning hole. The shaft 30 is connected to the bearing 26, and the flat head 31 on the shaft 30 extends out of the positioning hole and is connected to the flat groove 108 on the valve core inside the mounting groove 2.

The two adjacent sides of the two bosses 5 are connected as a whole, and a sensor is provided on the outer side of each of the two bosses 5, which detects the position of the coupling shaft, and judges the position or the opening and closing of the valve core by the position of the coupling shaft.

As a further preferred embodiment, the structure of the sensor is a U-shaped groove 7 facing the opening of the boss 5 so that the positioning block 29 can enter and exit the U-shaped groove 7 during rotation. The U-shaped groove 7 is composed of two parallel sides and a connecting side connecting the two parallel sides, in which one of the parallel edges is fixedly connected to the back of the pump body 1. When the coupling shaft 27 is mounted, the side of the positioning block 29 extending out of the body 28 faces the sensor during its rotation on the boss 5. During the rotation of the coupling shaft 27, the positioning block 29 extending out of the body 28 will enter or exit the sensor with the U-shaped groove 7 structure, and the signal of the entry and exit of the positioning block 29 can be detected by the sensor, which is connected to the circuit board 8 to enable signal transmission.

Preferably, when the positioning block 29 enters the sensor, one side of the positioning block 29 is in contact with a right-angle side surface of the right-angle convex surface 6 exactly to achieve better positioning effect and higher accuracy. The location of the sensor and several right-angle convex surfaces 6 is shown in FIG. 19.

As a further preferred embodiment, the mounting groove 2 is a structure with an opening on the top and a necking on the bottom. The opening is used for placing the two cylindrical structures and two input pipes of the control valve 10, and the necking for placing the output pipe 115; On the two side walls of the lower end of the opening is provided with an arc-shaped fixing groove 9 that accommodates the first cylindrical structure 111 and the second cylindrical structure 112 exactly. When the control valve 10 is in the mounting groove 2, the outer sides of the first cylindrical structure and the second cylindrical structure are clamped in the fixing grooves 9 on both sides, so that the control valve 10 can be better fixed in the mounting groove 2.

In addition, in this example, the motor and the coupling shaft may not be used, and the valve core can be directly manually screwed to achieve control. Of course, out of considerations such as saving manpower, material resources, and accuracy, it is preferable to adopt a motor control method.

### Example 4

As shown in FIG. 21, the specific working process of the nutrition pump is as follows: the valve core is mounted inside the cylindrical structure with the notch of the valve core aligned with that of the cylindrical structure; the coupling shaft 27 is connected to the valve core, and two DC motors which are respectively connected to the corresponding coupling shafts drive the corresponding valve cores to make a synchronous rotation movement inside the cylindrical structure through the corresponding coupling shafts, so as to realize the alternate output of the nutrient solution 13 and the cleaning solution 14. Specifically, when the nutrient solution is needed, opening the valve core corresponding to the nutrient solution and making the nutrient solution pass through the valve core and enter an output pipe; when the cleaning solution is needed, first closing the valve core corresponding to the nutrient solution, and then opening the cleaning solution valve core so that the cleaning solution enters the output pipe through the valve core. In this way, liquid mixing can be absolutely avoided.

Since the positioning block of the coupling shaft can enter and exit the sensor during its rotation, the position of the coupling shaft can be detected, which reflects the position of the valve core.

1) If the output result of the sensor is that the valve core of the cleaning solution is closed and the valve core of the nutrient solution is opened, the DC motors 35a and 35b will not rotate, and the rotation of the pump wheel 34 is controlled to realize the infusion of nutrient solution;

2) If the valve core of the cleaning solution is closed and the valve core of the nutrient solution is closed, the DC motor 35a connected to the valve core of the nutrient solution is controlled to rotate for a certain period of time to open the valve core of the nutrient solution, and then the signal from the detection sensor 7a on the side of the nutrient solution is detected; if the valve core of the nutrient solution is opened, the pump wheel is controlled to rotate to realize the infusion of the nutrient solution; if the valve core of the nutrient solution is still closed, an alarm will be issued;

3) If the valve core of the cleaning solution is opened and the valve core of the nutrient solution is closed, firstly the DC motor 35b connected to the valve core of the cleaning solution is controlled to rotate for a certain period of time to close the valve core of the cleaning solution, and then the signal from the detection sensor 7b on the side of the cleaning solution is detected; if the valve core of the cleaning solution is closed, the pump wheel is controlled to rotate to realize the infusion of the nutrient solution; if the valve core of the cleaning solution is still opened, an alarm will be issued;

4) If there is a state where the valve cores of the cleaning solution and the nutrient solution is both opened, a malposition alarm will be issued.

In the present disclosure, the nutrient solution bag 13 and the cleaning solution bag 14 can be replaced with two piercers 24, and the piercer 24 can be used with different medical solutions, nutrient solutions and/or cleaning solutions, etc., to achieve the infusion of different medical solutions.

The above are only the preferred specific examples of the present disclosure; however, the protection scope of the present disclosure is not limited thereto. Variations or substitutions those any person skilled in the art can easily think of within the technical scope disclosed in the present disclosure are all encompassed in the protection scope of the present disclosure. Therefore, the protection scope of the present disclosure should be subject to the protection scope of the claims.

1. A nutrition pump, comprising a pump body and an infusion set connectable to the pump body, in which the infusion set comprising a control valve, and the pump body is mounted with a pump wheel, wherein:
the control valve comprises a valve body and a valve core capable of matingly connecting with the valve body, the valve body comprises a first input pipe and a second input pipe, a cylindrical structure, and an output pipe for outputting liquid, the cylindrical structure is composed of a first cylindrical structure and a second cylindrical structure arranged side by side;
the valve core comprises a first valve core capable of being exactly accommodated in the first cylindrical structure and rotating in the first cylindrical structure, and a second valve core capable of being exactly accommodated in the second cylindrical structure and rotating in the second cylindrical structure;
the side wall of the first cylindrical structure is provided with a first through hole communicating with the first input pipe and a second through hole communicating with the output pipe, respectively, and correspondingly, the side wall of the second cylindrical structure is provided with a third through hole communicating with the second input pipe and a fourth through hole communicating with the output pipe, respectively;
the side wall of the first valve core is provided with a first hole and a second hole that communicate with each other, and the side wall of the second valve core is provided with a third hole and a fourth hole that communicate with each other;
when the two through holes on the cylindrical structure and the two holes on the valve core are aligned one by one, the liquid enters the output pipe through the valve core, and the valve core is opened; when the two through holes on the cylindrical structure are staggered with the two holes on the valve core, the liquid in the input pipe cannot enter the output pipe, and the valve core is closed.

2. The nutrition pump according to claim 1, wherein the first valve core is provided with a bent first communicating pipe that communicates the first hole and the second hole to allow liquid to pass through; and the second valve core is also provided with a bent second communicating pipe connecting the third hole and the fourth hole to allow the liquid to pass through.

3. The nutrition pump according to claim 1, wherein the first hole and the second hole on the first valve core are two independent through holes distributed at 90 degrees, and the third hole and the fourth hole on the second valve core are also two independent through holes distributed at 90 degrees;
correspondingly, the first through hole and the second through hole on the first cylindrical structure are two independent through holes distributed at 90 degrees, and the third through hole and the fourth through hole on the second cylindrical structure are also two independent through holes distributed at 90 degrees.

4. The nutrition pump according to claim 1, wherein the first cylindrical structure and the second cylindrical structure are both horizontally arranged in a front-to-rear direction, and the first input pipe and the second input pipe are respectively arranged vertically on the top of the first cylindrical structure and the top of the second cylindrical structure, and respectively communicate with the first through hole and the third through hole;
the output pipe is vertically arranged at the lower centers of the first cylindrical structure and the second cylindrical structure, and the top end of the output pipe is located between the first cylindrical structure and the second cylindrical structure.

5. The nutrition pump according to claim 4, wherein the first cylindrical structure and the second cylindrical structure are both cylinder-shaped structures with one end opened and the other closed, and at least one annular groove is provided on the inner walls of the first cylindrical structure and the second cylindrical structure;
the outer peripheries of the first valve core and the second valve core are both provided with an annular protruding strip matched with the annular groove so that the valve core can be fixedly connected in the cylindrical structure and can rotate freely in the cylindrical structure.

6. The nutrition pump according to claim 1, wherein the infusion set further comprises a first upper pipe, a second upper pipe, and a lower pipe, and the first input pipe in the control valve is connected to the first upper pipe used for transporting liquid, and the second upper pipe is connected to the second input pipe used for transporting liquid, and the output pipe is connected to the down pipe to output the liquid from the down pipe.

7. The nutrient pump according to claim 6, wherein the first cylindrical structure and the second cylindrical structure are each provided with a first notch at the openings on the walls, and the first valve core and the second valve core are each provided with a second notch at the openings on the walls, and the first notch on the first cylindrical structure and the second cylindrical structure are respectively aligned with the second notch on the first valve core and the second valve core in the initial state.

8. The nutrition pump according to claim 1, wherein the pump body is also mounted with a motor and a coupling shaft; one end of the coupling shaft is connected to the motor, and the other end is connected to the valve core so that the motor drives the valve core to rotate through the coupling shaft;
the coupling shaft comprises a main body, the top end surface of the main body is detachably connected to the motor, a positioning block protruding from the bottom end surface is transversely provided in the center of the bottom end surface of the main body, and one end of the positioning block extends outward to outer side the body, and a shaft is fixed on the lower end surface of the positioning block.

9. The nutrition pump according to claim 1, wherein the valve body further comprises a horizontally arranged flat plate, one end of the flat plate is fixedly connected to the outer peripheries of the first input pipe and the second input pipe and extends backward to the outer sides of the first cylindrical structure and the second cylindrical structure, and the bottom surface of the flat plate is fixedly connected to the top surfaces of the first cylindrical structure and the second cylindrical structure;
the lower end of the flat plate located above the output pipe extends downward and is fixedly connected to the top of the output pipe, and the upper end of the output pipe is located between the first cylindrical structure and the second cylindrical structure and is fixedly connected to the two cylindrical structures so that the control valve is integrated as a whole.

10. The nutrition pump according to claim 1, wherein the pump body comprises a front surface and a back surface, the upper end of the front surface is provided with a mounting groove for mounting the control valve, and the back surface is provided in a place corresponding to the mounting groove with two circular bosses for placing the coupling shaft, the centers of each boss are provided with a fixing hole for communicating with the mounting groove, and the shaft of the coupling shaft passes through the fixing hole from the back of the body and connects with the valve core in the mounting groove.

11. The nutrition pump according to claim 10, wherein each of the bosses is provided with two center-symmetric or axisymmetric right-angle convex surfaces, the edges at the right angles of the right-angle convex surfaces are flush with the inner side of the fixing hole;
the shaft is arranged in the positioning hole, the positioning block is placed on the upper end surface of the boss and can be rotated on the upper end surface of the boss with the shaft as a rotation axis, and the two right-angle surfaces of the right-angle convex surface can control the positioning block only to be rotated clockwise or counterclockwise within 90 degrees.

12. The nutrition pump according to claim 10, wherein a sensor for detecting the position of the coupling shaft is provided on the outer side of each of the two bosses to determine the opening and closing of the valve core.

13. The nutrition pump according to claim 11, wherein the walls on both sides of the lower end of the mounting groove are provided with arc-shaped fixing grooves that can accommodate the first cylindrical structure and the second cylindrical structure exactly, so that the outer side of the first cylindrical structure and the outer side of the second cylindrical structure are exactly clamped in the fixing grooves on both sides when the control valve is in the mounting groove;
the centers of one end surface of the first valve core and of the second valve core are each provided with a flat groove; the second notch and the flat groove are both located on the front side of the valve core, and the second notch communicates with the flat groove and has a depth smaller than that of the flat groove;
the free end of the shaft is a flat head for extending into the flat groove of the valve core to drive the valve core to rotate;
the upper end of the front surface of the main body is also provided with a pipe groove for placing the down pipe and the like, and the pump wheel is arranged at the lower end of the front surface of the main body.

14. (canceled)

15. A control valve according to claim 1.

16. A method for controlling liquid, comprising:
mounting a valve core inside a cylindrical structure with a second notch on the valve core aligned with a first notch of the cylindrical structure; placing a positioning block of a coupling shaft on a boss, and making a shaft of the coupling shaft pass through a fixing hole from the back of a pump body into a mounting groove and connect with the valve core in the mounting groove, connecting two DC motors to the corresponding coupling shafts respectively so that the DC motors drive the corresponding valve cores through the corresponding coupling shafts to rotate synchronously inside the cylindrical structure, so as to realize the alternate output of the nutrient solution and the cleaning solution; and detecting the position of the coupling shaft using a sensor and transmitting it to a circuit board for control;
specifically, when the nutrient solution is needed, opening the valve core corresponding to the nutrient solution and making the nutrient solution pass through the valve core and enter an output pipe; when the cleaning solution is needed, first closing the valve core corresponding to the nutrient solution, and then opening the cleaning solution valve core so that the cleaning solution enters the output pipe through the valve core.

17. The method according to claim 16, wherein the method further includes the following situations:
\textcircled{1} If the output result of the detection sensor is that the valve core of the cleaning solution is closed and the valve core of the nutrient solution is opened, the DC motor will not rotate, and the rotation of the pump wheel is controlled to realize the infusion of nutrient solution;
\textcircled{2} If the valve core of the cleaning solution is closed and the valve core of the nutrient solution is closed, the DC motor connected to the valve core of the nutrient solution is controlled to rotate for a certain period of time to open the valve core of the nutrient solution, and then the signal from the detection sensor on the side of the nutrient solution is detected; if the valve core of the nutrient solution is opened, the pump wheel is controlled to rotate to realize the infusion of the nutrient solution; if the valve core of the nutrient solution is still closed, an alarm will be issued;
\textcircled{3} If the valve core of the cleaning solution is opened and the valve core of the nutrient solution is closed, firstly the DC motor connected to the valve core of the cleaning solution is controlled to rotate for a certain period of time to close the valve core of the cleaning solution, and then the signal from the detection sensor on the side of the cleaning solution is detected; if the valve core of the cleaning solution is closed, the pump wheel is controlled to rotate to realize the infusion of the nutrient solution; if the valve core of the cleaning solution is still opened, an alarm will be issued;
\textcircled{4} If there is a state where the valve cores of the cleaning solution and the nutrient solution is both opened, an malposition alarm will be issued.
